(19) United States
(12) Patent Application Publication
(10) Pub. No.: US 2025/0225655 A1
(43) Pub. Date: Jul. 10, 2025

(54) BLOOD VESSEL SEGMENT DISCRIMINATION SYSTEM, BLOOD VESSEL SEGMENT DISCRIMINATION METHOD, AND PROGRAM

(71) Applicant: TEIKYO UNIVERSITY, Tokyo (JP)

(72) Inventor: Takeshi Takata, Tokyo (JP)

(21) Appl. No.: 18/852,886

(22) PCT Filed: Mar. 2, 2023
(86) PCT No.: PCT/JP2023/007803

§ 371 Date: Sep. 30, 2024

(30) Foreign Application Priority Data
Apr. 5, 2022 (JP) 2022-062934

(51) Int. Cl. G06T 7/00; A61B 5/00; A61B 5/055; A61B 6/00; A61B 6/03; G06T 7/11; G06T 7/149; G06T 11/00; G06V 10/774; G06V 20/64

(52) U.S. Cl. CPC G06T 7/0012; A61B 5/004; A61B 5/055; A61B 6/032; A61B 6/5205; G06T 7/11; G06T 7/149; G06T 11/006; G06T 11/008; G06V 10/774; G06V 20/64; G06T 2200/04; G06T 2207/10028; G06T 2207/10088; G06T 2207/20081; G06T 2207/30101; G06T 2207/30196; G06T 2207/30204; G06T 2211/441; G06V 2201/03

(57) ABSTRACT

A blood vessel segment discrimination system recognizes a three-dimensional structure of an abdomen of the patient, generates a depth image of the abdomen of the patient from the three-dimensional structure of the abdomen of the patient, generates a training data set used for training of a deep learning model, and discriminates an aortic segment of the patient using a trained deep learning model. A training data set generation unit generates the three-dimensional structure of the abdominal surface for learning from an abdominal CT image or the like of a person different from a patient who is a discrimination target of the aortic segment by the blood vessel segment discrimination device, generates a depth image for training from the three-dimensional structure of the abdominal surface for training, and generates a training data set showing a correspondence relationship between each pixel in the depth image for training and any of an aortic Zone 1, an aortic Zone 2, an aortic Zone 3, and another segment other than those Zones, based on the abdominal CT image.

### TECHNICAL FIELD

The present invention relates to a blood vessel segment discrimination system, a blood vessel segment discrimination method, and a program.

Priority is claimed on Japanese Patent Application No. 2022-062934, filed in Japan on Apr. 5, 2022, the content of which is incorporated herein by reference.

### BACKGROUND ART

Resuscitative endovascular balloon occlusion of the aorta (REBOA) is a critical care technique in which a balloon catheter is used to perform hemostasis or control the amount of bleeding for hemodynamics unstable traumatic bleeding or the like. In REBOA, a balloon is placed in the aorta using a catheter. REBOA is also performed in situations in which an X-ray fluoroscopy device, an ultrasound device, or the like cannot be used, unlike normal catheter treatment. In recent years, REBOA has also attracted attention as an effective technique for non-traumatic bleeding as well as traumatic bleeding.

In REBOA, perfusion of a non-bleeding part can be selectively preserved by adjusting a Zone 1 to a Zone 3 of the aorta in which the balloon is placed (Zone 1 to Zone 3 of the aorta are segmented according to branching positions of the aorta). Zone 1 to Zone 3 of the aorta are described in, for example, Non-Patent Document 1.

In a situation in which an X-ray fluoroscopy device, an ultrasound device, or the like can be used, and a catheter and Zone 1 to Zone 3 of the aorta can be observed by the X-ray fluoroscopy device, the ultrasound device, or the like, a position of the catheter with respect to Zone 1 to Zone 3 of the aorta is adjusted while checking the position of the catheter and positions of Zone 1 to Zone 3 of the aorta.

For example, even in a situation in which an X-ray fluoroscopy device, an ultrasound device, or the like cannot be used, such as in an emergency, and Zone 1 to Zone 3 of the aorta cannot be observed by the X-ray fluoroscopy device, the ultrasound device, or the like, REBOA may be performed.

In such a case, in the related art, an operator is required to perform REBOA in a state in which Zone 1 to Zone 3 of the aorta of a patient cannot be ascertained.

### CITATION LIST

### Non-Patent Documents

### Non-Patent Document 1:

Markus Harboe Olsen et al., “Standardized distances for placement of REBOA in patients with aortic stenosis”, Scientific Reports, volume 10, Article number: 13410 (2020)

### Non-Patent Document 2:

Takeshi Takata, Susumu Nakabayashi, Hiroshi Kondo, Masayoshi Yamamoto, Shigeru Furui, Kenshiro Shiraishi, Takenori Kobayashi, Hiroshi Oba, Takahide Okamoto & Jun'ichi Kotoku “Mixed Reality Visualization of Radiation Dose for Health Professionals and Patients in Interventional Radiology” Journal of Medical Systems 45:38 (2021)

### Non-Patent Document 3:

Emily Rae, Andras Lasso, Matthew S. Holden, Evelyn Morin, Ron Levy & Gabor Fichtinger “Neurosurgical burr hole placement using the Microsoft HoloLens” Medical Imaging 2018: Image-Guided Procedures, Robotic Interventions, and Modeling

### SUMMARY OF INVENTION

### Technical Problem

In view of the above, an object of the present invention is to provide a blood vessel segment discrimination system, a blood vessel segment discrimination method, and a program, which enable easy identification of a Zone 1, a Zone 2, and a Zone 3 of the aorta of a patient and can support implementation of REBOA even in a situation in which an X-ray fluoroscopy or ultrasound device cannot be used.

### Solution to Problem

According to an aspect of the present invention, a blood vessel segment discrimination system is provided including: a three-dimensional structure recognition device configured to recognize a three-dimensional structure of an abdomen of the patient; a depth image generation unit configured to generate a depth image of the abdomen of the patient from the three-dimensional structure of the abdominal surface of the patient recognized by the three-dimensional structure recognition device; a blood vessel segment discrimination device configured to discriminate an aortic segment of the patient whose three-dimensional structure of the abdominal surface is recognized by the three-dimensional structure recognition device, using a deep learning model; and a training data set generation unit configured to generate a training data set used for training of the deep learning model, in which, before the training of the deep learning model is performed, the training data set generation unit generates the three-dimensional structure of the abdominal surface for training from any of an abdominal computed tomography (CT) image, an abdominal magnetic resonance imaging (MRI) image, and an abdominal magnetic resonance angiography (MRA) image of a person different from a patient who is a discrimination target of the aortic segment by the blood vessel segment discrimination device, generates a depth image for training from the three-dimensional structure of the abdominal surface for training, and generates the training data set showing a correspondence relationship between each pixel in the depth image for training and any of a first blood vessel segment corresponding to a Zone 1 of an aorta, a second blood vessel segment corresponding to a Zone 2 of the aorta, a third blood vessel segment corresponding to a Zone 3 of the aorta, and another segment, based on any of the abdominal CT image, the abdominal MRI image, and the abdominal MRA image, and after the training of the deep learning model is performed using the training data set generated by the training data set generation unit, the three-dimensional structure recognition device recognizes the three-dimensional structure of the abdomen of the patient, the depth image generation unit generates the depth image of the abdomen of the patient from the three-dimensional structure of the abdominal surface of the patient recognized by the three-dimensional structure recognition device, and the blood vessel segment discrimination device estimates whether each pixel in the depth image of the abdomen of the patient generated by the depth image generation unit corresponds to any of the first blood vessel segment, the second blood vessel segment, the third blood vessel segment, and another segment using the trained deep learning model.

According to an aspect of the present invention, a blood vessel segment discrimination method for a blood vessel segment discrimination system is provided including: a three-dimensional structure recognition device configured to recognize a three-dimensional structure of an abdomen of the patient; a depth image generation unit configured to generate a depth image of the abdomen of the patient from the three-dimensional structure of the abdominal surface of the patient recognized by the three-dimensional structure recognition device; a blood vessel segment discrimination device configured to discriminate an aortic segment of the patient whose three-dimensional structure of the abdominal surface is recognized by the three-dimensional structure recognition device, using a deep learning model; and a training data set generation unit configured to generate a training data set used for training of the deep learning model, the blood vessel segment discrimination method including: a training data set generation step of, before the training of the deep learning model is performed, via the training data set generation unit, generating the three-dimensional structure of the abdominal surface for training from any of an abdominal CT image, an abdominal MRI image, and an abdominal MRA image of a person different from a patient who is a discrimination target of the aortic segment by the blood vessel segment discrimination device, generating a depth image for training from the three-dimensional structure of the abdominal surface for training, and generating the training data set showing a correspondence relationship between each pixel in the depth image for training and any of a first blood vessel segment corresponding to a Zone 1 of an aorta, a second blood vessel segment corresponding to a Zone 2 of the aorta, a third blood vessel segment corresponding to a Zone 3 of the aorta, and another segment, based on any of the abdominal CT image, the abdominal MRI image, and the abdominal MRA image; a three-dimensional structure recognition step of, after the training of the deep learning model is performed using the training data set generated in the training data set generation step, via the three-dimensional structure recognition device, recognizing the three-dimensional structure of the abdomen of the patient; a depth image generation step of, via the depth image generation unit, generating the depth image of the abdomen of the patient from the three-dimensional structure of the abdominal surface of the patient recognized in the three-dimensional structure recognition step; and a blood vessel segment discrimination step of, via the blood vessel segment discrimination device, estimating whether each pixel in the depth image of the abdomen of the patient generated in the depth image generation step corresponds to any of the first blood vessel segment, the second blood vessel segment, the third blood vessel segment, and another segment using the trained deep learning model.

According to an aspect of the present invention, a program for causing a computer constituting a blood vessel segment discrimination device provided in a blood vessel segment discrimination system is provided including: a three-dimensional structure recognition device configured to recognize a three-dimensional structure of an abdomen of the patient; a depth image generation unit configured to generate a depth image of the abdomen of the patient from the three-dimensional structure of the abdominal surface of the patient recognized by the three-dimensional structure recognition device; and a training data set generation unit configured to generate a training data set used for training of a deep learning model, to execute: a training step of performing the training of the deep learning model using the training data set generated by the training data set generation unit; and a blood vessel segment discrimination step, in which the training data set generation unit generates the three- dimensional structure of the abdominal surface for training from any of an abdominal CT image, an abdominal MRI image, and an abdominal MRA image of a person different from a patient who is a discrimination target of an aortic segment by the blood vessel segment discrimination device, generates a depth image for training from the three-dimensional structure of the abdominal surface for training, and generates the training data set showing a correspondence relationship between each pixel in the depth image for training and any of a first blood vessel segment corresponding to a Zone 1 of an aorta, a second blood vessel segment corresponding to a Zone 2 of the aorta, a third blood vessel segment corresponding to a Zone 3 of the aorta, and another segment, based on any of the abdominal CT image, the abdominal MRI image, and the abdominal MRA image, after the training step is executed, the three-dimensional structure recognition device recognizes the three-dimensional structure of the abdomen of the patient, and the depth image generation unit generates the depth image of the abdomen of the patient from the three-dimensional structure of the abdominal surface of the patient recognized by the three-dimensional structure recognition device, and in the blood vessel segment discrimination step, estimation is made as to whether each pixel in the depth image of the abdomen of the patient generated by the depth image generation unit corresponds to any of the first blood vessel segment, the second blood vessel segment, the third blood vessel segment, and another segment using the trained deep learning model.

According to an aspect of the present invention, a blood vessel segment discrimination system is provided including: a three-dimensional structure recognition device configured to recognize a three-dimensional structure of an abdomen of the patient; a blood vessel segment discrimination device configured to discriminate an aortic segment of the patient whose three-dimensional structure of the abdominal surface is recognized by the three-dimensional structure recognition device, using a deep learning model; and a training data set generation unit configured to generate a training data set used for training of the deep learning model, in which, before the training of the deep learning model is performed, the training data set generation unit, generates the three-dimensional structure of the abdominal surface for training from any of an abdominal CT image, an abdominal MRI image, and an abdominal MRA image of a person different from a patient who is a discrimination target of the aortic segment by the blood vessel segment discrimination device, and generates the training data set showing a correspondence relationship between each point on the three-dimensional structure of the abdominal surface for training and any of a first blood vessel segment corresponding to a Zone 1 of an aorta, a second blood vessel segment corresponding to a Zone 2 of the aorta, a third blood vessel segment corresponding to a Zone 3 of the aorta, and another segment, based on any of the abdominal CT image, the abdominal MRI image, and the abdominal MRA image, and after the training of the deep learning model is performed using the training data set generated by the training data set generation unit, the three-dimensional structure recognition device recognizes the three-dimensional structure of the abdomen of the patient, and the blood vessel segment discrimination device estimates whether each point on the three-dimensional structure of the abdomen of the patient recognized by the three-dimensional structure recognition device corresponds to any of the first blood vessel segment, the second blood vessel segment, the third blood vessel segment, and another segment using the trained deep learning model.

According to an aspect of the present invention, a blood vessel segment discrimination method for a blood vessel segment discrimination system is provided including: a three-dimensional structure recognition device configured to recognize a three-dimensional structure of an abdomen of the patient; a blood vessel segment discrimination device configured to discriminate an aortic segment of the patient whose three-dimensional structure of the abdominal surface is recognized by the three-dimensional structure recognition device, using a deep learning model; and a training data set generation unit configured to generate a training data set used for training of the deep learning model, the blood vessel segment discrimination method including: a training data set generation step of, before the training of the deep learning model is performed, via the training data set generation unit, generating the three-dimensional structure of the abdominal surface for training from any of an abdominal CT image, an abdominal MRI image, and an abdominal MRA image of a person different from a patient who is a discrimination target of the aortic segment by the blood vessel segment discrimination device, and generating the training data set showing a correspondence relationship between each point on the three-dimensional structure of the abdominal surface for training and any of a first blood vessel segment corresponding to a Zone 1 of an aorta, a second blood vessel segment corresponding to a Zone 2 of the aorta, a third blood vessel segment corresponding to a Zone 3 of the aorta, and another segment, based on any of the abdominal CT image, the abdominal MRI image, and the abdominal MRA image; a three-dimensional structure recognition step of, after the training of the deep learning model is performed using the training data set generated in the training data set generation step, via the three-dimensional structure recognition device, recognizing the three-dimensional structure of the abdomen of the patient; and a blood vessel segment discrimination step of, via the blood vessel segment discrimination device, estimating whether each point on the three-dimensional structure of the abdomen of the patient recognized in the three-dimensional structure recognition step corresponds to any of the first blood vessel segment, the second blood vessel segment, the third blood vessel segment, and another segment using the trained deep learning model.

According to an aspect of the present invention, a program for causing a computer constituting a blood vessel segment discrimination device provided in a blood vessel segment discrimination system is provided including: a three-dimensional structure recognition device configured to recognize a three-dimensional structure of an abdomen of the patient; and a training data set generation unit configured to generate a training data set used for training of a deep learning model, to execute: a training step of performing the training of the deep learning model using the training data set generated by the training data set generation unit; and a blood vessel segment discrimination step, in which the training data set generation unit generates the three-dimensional structure of the abdominal surface for training from any of an abdominal CT image, an abdominal MRI image, and an abdominal MRA image of a person different from a patient who is a discrimination target of an aortic segment by the blood vessel segment discrimination device, and generates the training data set showing a correspondence relationship between each point on the three-dimensional structure of the abdominal surface for training and any of a first blood vessel segment corresponding to a Zone 1 of an aorta, a second blood vessel segment corresponding to a Zone 2 of the aorta, a third blood vessel segment corresponding to a Zone 3 of the aorta, and another segment, based on any of the abdominal CT image, the abdominal MRI image, and the abdominal MRA image, after the training step is executed, the three-dimensional structure recognition device recognizes the three-dimensional structure of the abdomen of the patient, and in the blood vessel segment discrimination step, estimation is made as to whether each point on the three-dimensional structure of the abdomen of the patient recognized by the three-dimensional structure recognition device corresponds to any of the first blood vessel segment, the second blood vessel segment, the third blood vessel segment, and another segment using the trained deep learning model.

### Advantageous Effects of Invention

According to the present invention, it is possible to provide a blood vessel segment discrimination system, a blood vessel segment discrimination method, and a program with which it is possible to easily identify a Zone 1, a Zone 2, and a Zone 3 of the aorta of a patient even in a situation in which an X-ray fluoroscopy or ultrasound device cannot be used, and it is possible to support the implementation of REBOA.

### BRIEF DESCRIPTION OF DRAWINGS

FIG. 1 is a diagram showing an example of a blood vessel segment discrimination system 1 according to a first embodiment.

FIG. 2 is a diagram describing an example of processing executed by a training data set generation unit 13.

FIG. 3 is a flowchart describing an example of processing executed in the blood vessel segment discrimination system 1 according to the first embodiment.

FIG. 4 is a diagram conceptually showing training of a semantic segmentation model using a training data set.

FIG. 5 is a diagram conceptually showing a test of a trained semantic segmentation model.

FIG. 6 is a diagram showing a result of evaluating, using a Dice coefficient and a Jaccard coefficient, a similarity between a Zone 1, a Zone 2, and a Zone 3 estimated using the trained semantic segmentation model.

FIG. 7 is a diagram showing a position error of a boundary line between Zone 1 and Zone 2, a position error of a boundary line between Zone 2 and Zone 3, and the like, which are estimated using the trained semantic segmentation model.

FIG. 8 is a diagram showing four examples in which a position error of an aortic segment estimated using the trained semantic segmentation model is less than 10 mm.

FIG. 9 is a diagram showing four examples in which a position error of an aortic segment estimated using the trained semantic segmentation model is larger than 20 mm.

FIG. 10 is a diagram conceptually describing a technique of projecting an estimation result of the Zone onto a body surface of a patient in real time using HoloLens 2.

FIG. 11 is a diagram showing an example of a blood vessel segment discrimination system 2 according to a third embodiment.

FIG. 12 is a flowchart describing an example of processing executed in the blood vessel segment discrimination system 2 according to the third embodiment.

### DESCRIPTION OF EMBODIMENTS

Hereinafter, embodiments of a blood vessel segment discrimination system, a blood vessel segment discrimination method, and a program according to the present invention will be described with reference to the drawings.

### First Embodiment

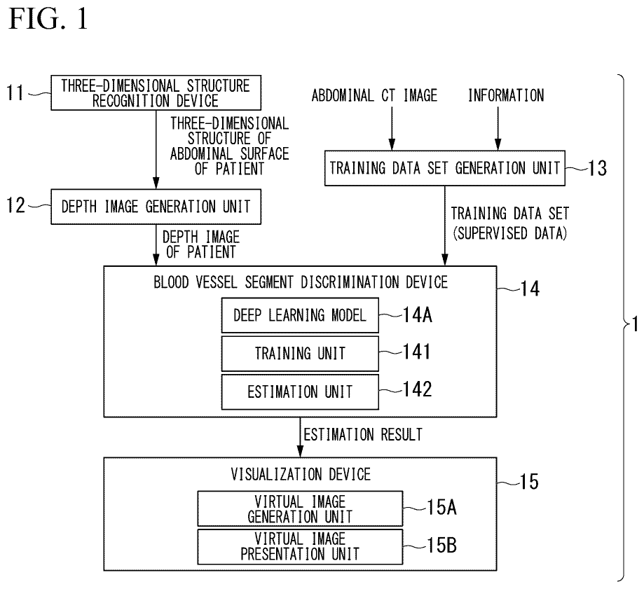

FIG. 1 is a diagram showing an example of a blood vessel segment discrimination system 1 according to a first embodiment.

In the example shown in FIG. 1, the blood vessel segment discrimination system 1 according to the first embodiment supports implementation of REBOA on a patient by an operator in a situation in which an X-ray fluoroscopy or ultrasound device cannot be used, for example, in an emergency. The blood vessel segment discrimination system 1 includes a three-dimensional structure recognition device 11, a depth image generation unit 12, a training data set generation unit 13, a blood vessel segment discrimination device 14, and a visualization device 15.

The three-dimensional structure recognition device 11 recognizes a three-dimensional structure of an abdomen of the patient. The three-dimensional structure recognition device 11 recognizes the three-dimensional structure of the abdomen of the patient and generates three-dimensional data of the abdomen of the patient using a technique similar to that used with a “LiDAR scanner” described on, for example, the following website.
https://bablishe.com/about-lidar-scanner-of-ipad-pro-2020/

As the three-dimensional structure recognition device 11, products described on, for example, the following websites and the like can be used.
https://www.apple.com/jp/ipad-pro/specs/
https://www.microsoft.com/ja-jp/hololens/hardware

The former product is used in a procedure described on, for example, the following website to recognize a three-dimensional structure of an external environment.
https://prono82.com/2020/09/28/apple%E7%A4%BElidar%E6%90%AD%E8%BC%89ipad-pro%E3%81%AB%E3%82%88%E3%82%8B3d%E7%82%B9%E7%BE%A4%E3%82%B9%E3% 82%AD%E3%83%A3%E3%83%B3%E3%82%A2%E3%83%97%E3%83%AA%E3%81%AE%E3%83%AA%E3%83%AA%E3%83%BC%E3%82%B9/

In addition, the latter product is used in a procedure described on, for example, the following websites to recognize a three-dimensional structure of external environment.
https://docs.microsoft.com/ja-jp/windows/mixed-reality/design/spatial-mapping
https://zenn.dev/hiromu/articles/20210421-scene-understanding

The depth image generation unit 12 generates a depth image of the abdomen of the patient from the three-dimensional structure of the abdominal surface of the patient recognized by the three-dimensional structure recognition device 11. The depth image is an image colored according to a distance, as described on, for example, the following website. That is, the depth image generation unit 12 generates a two-dimensional image of the abdomen of the patient in which the abdomen of the patient is colored according to a distance from the three-dimensional structure recognition device 11.

The depth image generation unit 12 generates the depth image of the abdomen of the patient using a technique described on, for example, the following website.
https://www.cit.nihon-u.ac.jp/laboratorydata/kenkyu/kouennkai/reference/No.47/pdf/2-53.pdf

The training data set generation unit 13 generates a training data set.

The blood vessel segment discrimination device 14 discriminates an aortic segment of the patient whose three-dimensional structure of the abdominal surface is recognized by the three-dimensional structure recognition device 11 using a deep learning model 14A.

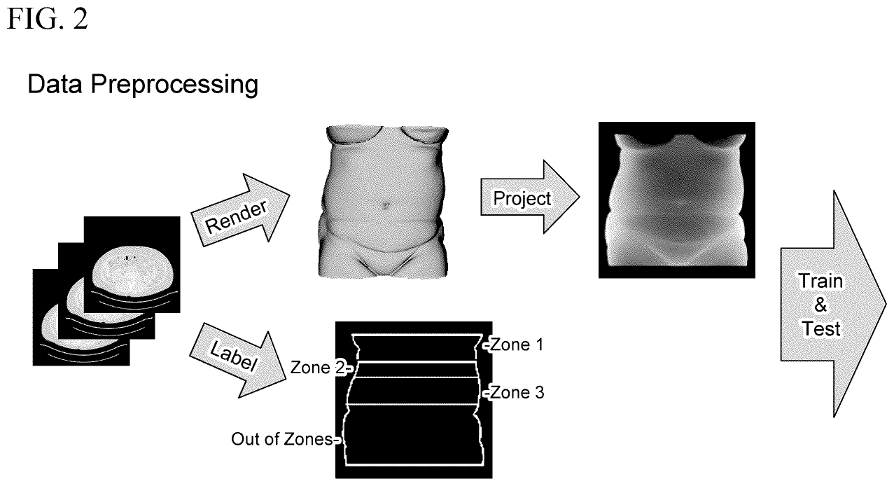

FIG. 2 is a diagram describing an example of processing executed by the training data set generation unit 13.

In the example shown in FIG. 2, the training data set generation unit 13 generates a three-dimensional structure (structure shown on the upper side of the center of FIG. 2) of an abdominal surface for training from an abdominal computed tomography (CT) image (image shown on the left side of FIG. 2) of a person different from a patient who is a discrimination target of the aortic segment by the blood vessel segment discrimination device 14. That is, the training data set generation unit 13 executes processing indicated by “Render” in FIG. 2. The three-dimensional structure of the abdominal surface for training generated in this processing is the same as a recognition result (three-dimensional structure) obtained in a case where the three-dimensional structure recognition device 11 recognizes the three-dimensional structure of the abdominal surface of the person (that is, a subject person of the abdominal CT image).

In addition, the training data set generation unit 13 generates a depth image for training (image shown on the upper right side of FIG. 2) from the three-dimensional structure of the abdominal surface for training generated by the processing indicated by “Render” in FIG. 2. That is, the training data set generation unit 13 executes processing indicated by “Project” in FIG. 2.

Further, the training data set generation unit 13 generates, based on the abdominal CT image (image shown on the left side of FIG. 2), information (information shown on the lower side of the center of FIG. 2) showing a correspondence relationship between each pixel in the depth image for training (image shown on the upper right side of FIG. 2) and any of a first blood vessel segment corresponding to a Zone 1 of the aorta, a second blood vessel segment corresponding to a Zone 2 of the aorta, a third blood vessel segment corresponding to a Zone 3 of the aorta, and another segment (a segment indicated by “Out of Zones” in FIG. 2). That is, the training data set generation unit 13 executes processing indicated by “Label” in FIG. 2.

In addition, the training data set generation unit 13 generates a training data set, which is a set of the depth image for training (image shown in the upper right side of FIG. 2) generated by the processing indicated by “Project” in FIG. 2 and the information (information shown in the lower side of the center of FIG. 2) generated by the processing indicated by “Label” in FIG. 2, as supervised data used for training the deep learning model 14A. In the example shown in FIG. 2, the abdominal CT image of a person different from a patient who is a discrimination target of the aortic segment by the blood vessel segment discrimination device 14 is used to generate the training data set. However, in another example, an abdominal magnetic resonance imaging (MRI) image of a person different from a patient who is a discrimination target of the aortic segment by the blood vessel segment discrimination device 14 may be used to generate the training data set, and in still another example, an abdominal magnetic resonance angiography (MRA) image of a person different from a patient who is a discrimination target of the aortic segment by the blood vessel segment discrimination device 14 may be used to generate the training data set.

In the example shown in FIG. 1, the blood vessel segment discrimination device 14 includes a training unit 141 and an estimation unit 142. The training unit 141 performs training of the deep learning model 14A using the training data set (for example, the image shown on the upper right side of FIG. 2 and the information shown on the lower side of the center of FIG. 2) generated by the training data set generation unit 13.

Specifically, since the risk of organ ischemia increases if an estimation accuracy of the second blood vessel segment corresponding to Zone 2 of the aorta is low in a case where REBOA is performed, the training unit 141 performs training of the deep learning model 14A such that the estimation accuracy of the second blood vessel segment corresponding to Zone 2 of the aorta using the trained deep learning model 14A is equal to or higher than a predetermined threshold value.

In the blood vessel segment discrimination system 1 according to the first embodiment, a segmentation model is used as the deep learning model 14A.

Specifically, in a first example of the blood vessel segment discrimination system 1 according to the first embodiment, as the deep learning model 14A, semantic segmentation models described on, for example, the following websites and the like are used.
https://jp.mathworks.com/content/dam/mathworks/mathworks-dot-com/company/events/webinar-cta/2459280_Basics_of_semantic_segmentation.pdf
https://qiita.com/fujiya228/items/ea30dac6ef827d608a56

In a second example of the blood vessel segment discrimination system 1 according to the first embodiment, as the deep learning model 14A, a segmentation model other than the semantic segmentation, such as instance segmentation and panoptic segmentation described on, for example, the following website, may be used.
https://www.skillupai.com/blog/tech/segmentation1/

In the example shown in FIG. 1, the estimation unit 142 uses the trained deep learning model 14A to estimate whether each pixel in the depth image of the abdomen of the patient generated by the depth image generation unit 12 corresponds to any of the first blood vessel segment corresponding to Zone 1 of the aorta, the second blood vessel segment corresponding to Zone 2 of the aorta, the third blood vessel segment corresponding to Zone 3 of the aorta, and another segment (that is, a segment that does not correspond to any of the first blood vessel segment, the second blood vessel segment, and the third blood vessel segment).

That is, in the example shown in FIG. 1, the three-dimensional structure recognition device 11 recognizes the three-dimensional structure of the abdomen of the patient after the deep learning model 14A is trained using the training data set generated by the training data set generation unit 13. In addition, the depth image generation unit 12 generates the depth image of the abdomen of the patient from the three-dimensional structure of the abdominal surface of the patient recognized by the three-dimensional structure recognition device 11. Further, the estimation unit 142 of the blood vessel segment discrimination device 14 uses the trained deep learning model 14A to estimate whether each pixel in the depth image of the abdomen of the patient generated by the depth image generation unit 12 corresponds to the first blood vessel segment corresponding to Zone 1 of the aorta, the second blood vessel segment corresponding to Zone 2 of the aorta, the third blood vessel segment corresponding to Zone 3 of the aorta, and another segment (a segment indicated by “Out of Zones” in FIG. 2).

The visualization device 15 generates a virtual image in which the estimation result of the aortic segment of the patient by the blood vessel segment discrimination device 14 is projected onto the abdomen of the patient. The visualization device 15 includes a virtual image generation unit 15A and a virtual image presentation unit 15B.

The virtual image generation unit 15A generates a virtual image in which the first blood vessel segment (Zone 1), the second blood vessel segment (Zone 2), and the third blood vessel segment (Zone 3) of the patient are projected onto the abdominal surface of the patient. The virtual image presentation unit 15B presents the virtual image generated by the virtual image generation unit 15A to a user (for example, an operator or the like) of the blood vessel segment discrimination system 1.

In the first example of the blood vessel segment discrimination system 1 according to the first embodiment, the visualization device 15 projects the first blood vessel segment (Zone 1), the second blood vessel segment (Zone 2), and the third blood vessel segment (Zone 3) of the patient onto the abdomen of the patient using the techniques described in, for example, Non-Patent Document 2, Non-Patent Document 3, and the following website.
https://prtimes.jp/main/html/rd/p/000000073.000004318.html

A projection technique of the first example of the blood vessel segment discrimination system 1 according to the first embodiment is, for example, a technique using an external marker as a reference, a technique of superimposing information on a human body by performing registration manually, or the like.

In the second example of the blood vessel segment discrimination system 1 according to the first embodiment, as the visualization device 15, a product described on, for example, the following websites is used.
https://www.apple.com/jp/ipad-pro/specs/

In the second example of the blood vessel segment discrimination system 1 according to the first embodiment, the visualization device 15 directly uses a real three-dimensional object (that is, the abdomen of the patient) as a marker using a technique similar to techniques described on, for example, the following websites, that is, directly projects the first blood vessel segment (Zone 1), the second blood vessel segment (Zone 2), and the third blood vessel segment (Zone 3) of the patient onto the abdomen of the patient without any external intervention.
https://library.vuforia.com/features/objects/object-reco.html
https://www.youtube.com/watch?v=jbaUDMvv2Zw

In a third example of the blood vessel segment discrimination system 1 according to the first embodiment, as the visualization device 15, a product described on, for example, the following website is used.
https://www.microsoft.com/ja-jp/hololens/hardware

In the third example of the blood vessel segment discrimination system 1 according to the first embodiment, the visualization device 15 directly uses a real three-dimensional object (that is, the abdomen of the patient) as a marker using a technique similar to techniques described on, for example, the following websites, that is, directly projects the first blood vessel segment (Zone 1), the second blood vessel segment (Zone 2), and the third blood vessel segment (Zone 3) of the patient onto the abdomen of the patient without any external intervention.
https://docs.microsoft.com/ja-jp/azure/object-anchors/overview
https://car.watch.impress.co.jp/docs/news/1278295.html
https://library.vuforia.com/features/objects/object-reco.html
https://www.youtube.com/watch?v=jbaUDMvv2Zw

In the third example of the blood vessel segment discrimination system 1 according to the first embodiment, the visualization device 15 generates a virtual image in which the first blood vessel segment (Zone 1), the second blood vessel segment (Zone 2), and the third blood vessel segment (Zone 3) of the patient are projected onto the abdominal surface of the patient using techniques similar to the techniques described on, for example, the following websites, and presents the virtual image to the user of the blood vessel segment discrimination system 1.
https://www.ogis-ri.co.jp/otc/hiroba/technical/point-DX/part6.html
https://www.tattichan.work/entry/2019/12/06/Reprojection%E3%81%AB%E3%81%A4%E3%81%84%E3%81%A6%E6%95%B4%E7%90%86%E3%81%99%E3%82%8B
https://www.businessinsider.jp/post-185961
https://hanada-sekkei.co.jp/armrvr
https://www.youtube.com/watch?v=QZiQ71EDF-o

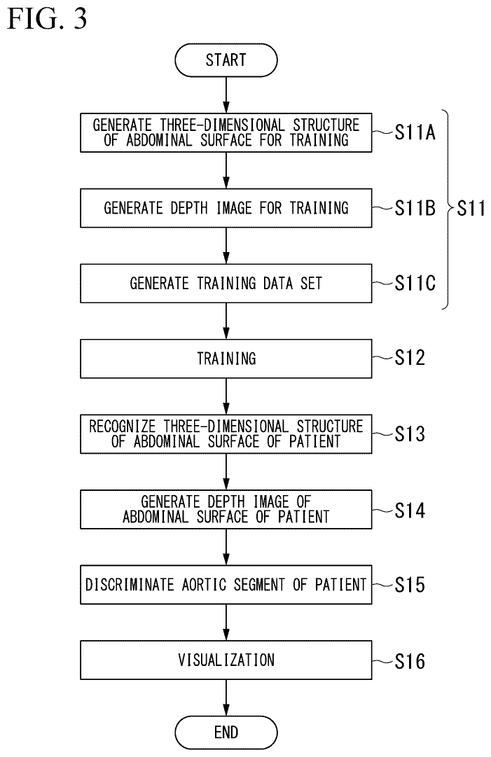

FIG. 3 is a flowchart describing an example of processing executed in the blood vessel segment discrimination system 1 according to the first embodiment.

In the example shown in FIG. 3, in step S11, the training data set generation unit 13 generates the training data set used for the training of the deep learning model 14A.

Specifically, in step S11A, the training data set generation unit 13 generates the three-dimensional structure of the abdominal surface for training from any of the abdominal CT image, the abdominal MRI image, and the abdominal MRA image of a person different from a patient who is a discrimination target of the aortic segment by the blood vessel segment discrimination device 14. Next, in step S11B, the training data set generation unit 13 generates the depth image for training from the three-dimensional structure of the abdominal surface for training generated in step S11A.

Next, in step S11C, the training data set generation unit 13 generates the training data set showing a correspondence relationship between each pixel in the depth image for training and any of the first blood vessel segment corresponding to Zone 1 of the aorta, the second blood vessel segment corresponding to Zone 2 of the aorta, the third blood vessel segment corresponding to Zone 3 of the aorta, and another segment (that is, a segment that does not correspond to any of the first blood vessel segment, the second blood vessel segment, and the third blood vessel segment), based on any of the abdominal CT image, the abdominal MRI image, and the abdominal MRA image.

Next, in step S12, the training unit 141 of the blood vessel segment discrimination device 14 performs training of the deep learning model 14A using the training data set generated in step S11.

Next, in step S13, the three-dimensional structure recognition device 11 recognizes the three-dimensional structure of the abdomen of the patient who is the discrimination target of the aortic segment by the blood vessel segment discrimination device 14.

Next, in step S14, the depth image generation unit 12 generates the depth image of the abdomen of the patient from the three-dimensional structure of the abdominal surface of the patient recognized in step S13.

Next, in step S15, the blood vessel segment discrimination device 14 discriminates the aortic segment of the patient whose three-dimensional structure of the abdominal surface is recognized in step S13 using the trained deep learning model 14A that has been trained in step S12.

Specifically, in step S15, the estimation unit 142 of the blood vessel segment discrimination device 14 uses the trained deep learning model 14A to estimate whether each pixel in the depth image of the abdomen of the patient generated in step S14 corresponds to any of the first blood vessel segment corresponding to Zone 1 of the aorta, the second blood vessel segment corresponding to Zone 2 of the aorta, the third blood vessel segment corresponding to Zone 3 of the aorta, and another segment (that is, a segment that does not correspond to any of the first blood vessel segment, the second blood vessel segment, and the third blood vessel segment).

Next, in step S16, the virtual image generation unit 15A of the visualization device 15 generates a virtual image in which the first blood vessel segment (Zone 1), the second blood vessel segment (Zone 2), and the third blood vessel segment (Zone 3) of the patient are projected onto the abdominal surface of the patient. In addition, the virtual image presentation unit 15B of the visualization device 15 presents the virtual image generated by the virtual image generation unit 15A to the user of the blood vessel segment discrimination system 1.

Therefore, with the blood vessel segment discrimination system 1 according to the first embodiment, the user of the blood vessel segment discrimination system 1 can easily identify Zone 1, Zone 2, and Zone 3 of the aorta of the patient even in a situation in which the X-ray fluoroscopy or ultrasound device cannot be used. As a result, it is possible to improve the availability of REBOA in a situation in which the X-ray fluoroscopy or ultrasound device cannot be used.

As described above, in the example shown in FIG. 1, the three-dimensional structure recognition device 11 recognizes the three-dimensional structure of the abdomen of the patient who is the discrimination target of the aortic segment by the blood vessel segment discrimination device 14 and generates the three-dimensional data of the abdomen of the patient using a technique similar to the LiDAR scanner.

In another example, as in the example shown in FIG. 2, the three-dimensional structure recognition device 11 may have a function of generating the three-dimensional structure of the abdomen of the patient from any of the abdominal CT image, the abdominal MRI image, and the abdominal MRA image of the patient who is the discrimination target of the aortic segment by the blood vessel segment discrimination device 14.

As described above, in the example shown in FIG. 1, the blood vessel segment discrimination device 14 outputs, as the estimation result of the aortic segment of the patient, information used by the visualization device 15 to generate the virtual image in which the first blood vessel segment (Zone 1), the second blood vessel segment (Zone 2), and the third blood vessel segment (Zone 3) are projected onto the abdomen of the patient.

In another example, the blood vessel segment discrimination device 14 may output, as the estimation result of the aortic segment of the patient, a length from a landmark part (that is, a site suitable for puncture (that is, a catheter insertion site)) of the patient to the first blood vessel segment (Zone 1) of the patient, a length from the landmark part of the patient to the second blood vessel segment (Zone 2) of the patient, and a length from the landmark part of the patient to the third blood vessel segment (Zone 3) of the patient, for example, in numerical values or like.

Further, in another example, as the visualization device 15, a product described on, for example, the following website is used.
https://www.apple.com/jp/ipad-pro/specs/

Further, in this example, the length from the landmark part (catheter insertion site) of the patient to the first blood vessel segment (Zone 1) of the patient, the length from the landmark part of the patient to the second blood vessel segment (Zone 2) of the patient, and the length from the landmark part of the patient to the third blood vessel segment (Zone 3) of the patient are measured using a technique described on the following website
https://support.apple.com/ja-jp/guide/ipad/ipad8ac2cfea/ipados

Specifically, in this example, the user of the blood vessel segment discrimination system 1 designates a point corresponding to the landmark part (catheter insertion site) of the patient and a point corresponding to, for example, the first blood vessel segment (Zone 1) of the patient on the virtual image presented to the user of the blood vessel segment discrimination system 1, whereby a length between these two points is measured and presented to the user of the blood vessel segment discrimination system 1. Therefore, the user of the blood vessel segment discrimination system 1 can determine a catheter length inserted into the patient using the presented result.

In addition, in another example, as the visualization device 15, a product described on, for example, the following website is used.
https://www.microsoft.com/ja-jp/hololens/hardware

Further, in this example, the length from the landmark part (catheter insertion site) of the patient to the first blood vessel segment (Zone 1) of the patient, the length from the landmark part of the patient to the second blood vessel segment (Zone 2) of the patient, and the length from the landmark part of the patient to the third blood vessel segment (Zone 3) of the patient are measured using a technique described on the following website.
https://www.windowscentral.com/hololens-gets-virtual-tape-measure

In this example as well, the user of the blood vessel segment discrimination system 1 designates a point corresponding to the landmark part (catheter insertion site) of the patient and a point corresponding to, for example, the first blood vessel segment (Zone 1) of the patient on the virtual image presented to the user of the blood vessel segment discrimination system 1, whereby a length between these two points is measured and presented to the user of the blood vessel segment discrimination system 1. Therefore, the user of the blood vessel segment discrimination system 1 can determine a catheter length inserted into the patient using the presented result.

In the two examples described above, as a balloon catheter to be inserted into the patient, a balloon catheter having length marks as described on, for example, the following website is used.
https://prytimemedical.com/product/er-reboa-plus-catheter/

In still another example, the training data set generation unit 13 may generate the training data set for learning a running state (meandering state) of a blood vessel into which the balloon catheter is inserted, based on any of the abdominal CT image (image shown on the left side of FIG. 2), the abdominal MRI image, and the abdominal MRA image.

In this example, the training unit 141 of the blood vessel segment discrimination device 14 performs training of the deep learning model 14A using the training data set. The estimation unit 142 of the blood vessel segment discrimination device 14 estimates a running state of the blood vessel of the patient into which the balloon catheter is inserted using the trained deep learning model 14A. Further, in a case where the user of the blood vessel segment discrimination system 1 designates a point corresponding to the landmark part (catheter insertion site) of the patient and a point corresponding to, for example, the first blood vessel segment (Zone 1) of the patient on the virtual image presented to the user of the blood vessel segment discrimination system 1, a length along the blood vessel between these two points is estimated and output based on a straight line distance between these two points and an estimation result of the estimation unit 142 of the blood vessel segment discrimination device 14.

In a modification example of this example, the training data set generation unit 13 may generate a training data set for learning a length along the blood vessel between the point corresponding to the landmark part (catheter insertion site) of the patient and the point corresponding to, for example, the first blood vessel segment (Zone 1) of the patient, based on any of the abdominal CT image (image shown on the left side of FIG. 2), the abdominal MRI image, and the abdominal MRA image.

In still another example, the visualization device 15 may generate a virtual image in which a balloon of the balloon catheter inserted into the blood vessel of the patient is in a state of being placed in any of the first blood vessel segment (Zone 1), the second blood vessel segment (Zone 2), and the third blood vessel segment (Zone 3) of the patient (that is, a state in which the balloon is expanded), and may present the virtual image to the user of the blood vessel segment discrimination system 1. In this example, the user of the blood vessel segment discrimination system 1 can easily imagine a state in which the balloon is actually placed in any of the first blood vessel segment (Zone 1), the second blood vessel segment (Zone 2), and the third blood vessel segment (Zone 3) of the patient.

### EXAMPLES

The present inventor researched and developed the deep learning model 14A that instantly discriminates the aortic segment from a body surface of a patient in testing the blood vessel segment discrimination system 1 of the first embodiment. Specifically, the present inventor estimated the aortic segment from three-dimensional information of the body surface of a patient by combining a segmentation technique based on advanced deep learning and depth information of the patient obtained from reflection of infrared light emitted from a camera corresponding to the three-dimensional structure recognition device 11.

The present inventor specializes in medical radiology technology, machine learning, and mixed reality, and has been conducting research on, for example, visualizing previously invisible radiation exposure through mixed reality. Medical applications in mixed reality have advanced, and mixed reality has started to play an active role in the three-dimensional visualization of organs and telemedicine. The present inventor considered that visualization of other unknown information in an intuitive manner could be of use in medical care support and has focused on the use of REBOA in life-saving medical care situations performed even when an X-ray fluoroscopy device is physically unusable. The present research is devised in consideration that estimation of a Zone segment to be visualized with high accuracy using deep learning technology enables visualization with high accuracy and immersiveness, which is significantly different from the related art and remarkably improves the quality of life-saving medical care.

The present inventor performed the following in order to construct a model that makes it possible to estimate the Zone using a technique of segmentation by deep learning and to establish a trained model that makes it possible to perform highly accurate estimation.

A training data set plays an extremely important role in performing deep learning, and the degree of completion of the training data set greatly affects the results of research. Therefore, the present inventor first focused on the preparation of a training data set. A body surface three-dimensional image created from the abdominal CT image was used for training. The present inventor mainly used data owned by Teikyo University Hospital as the CT image. Further, in order to avoid any bias of training data, the present inventor also utilized an open access CT image database. The present inventor, who is also a radiological technologist, performed segmentation of the training data and used the segmented training data as a training label. Further, the present inventor collected information on the age and the body weight in preparation for a case where the prediction accuracy was insufficient with only a body surface image, and added the information to the training data as necessary.

The present inventor performed training of a semantic segmentation model using a training data set. The present inventor appropriately performed a search for a hyperparameter and a modification of a network structure in the training to construct an optimal training model. The present inventor comprehensively evaluated the trained model using reproducibility, precision, a dice coefficient, and the like. Among these, particularly, the reproducibility of Zone 2 having a high risk of organ ischemia was focused on, aiming at the creation of a model capable of discriminating a high-risk region with high accuracy.

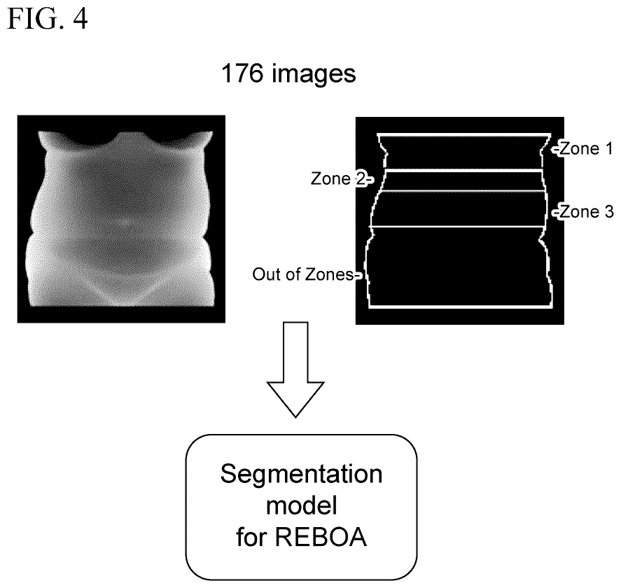

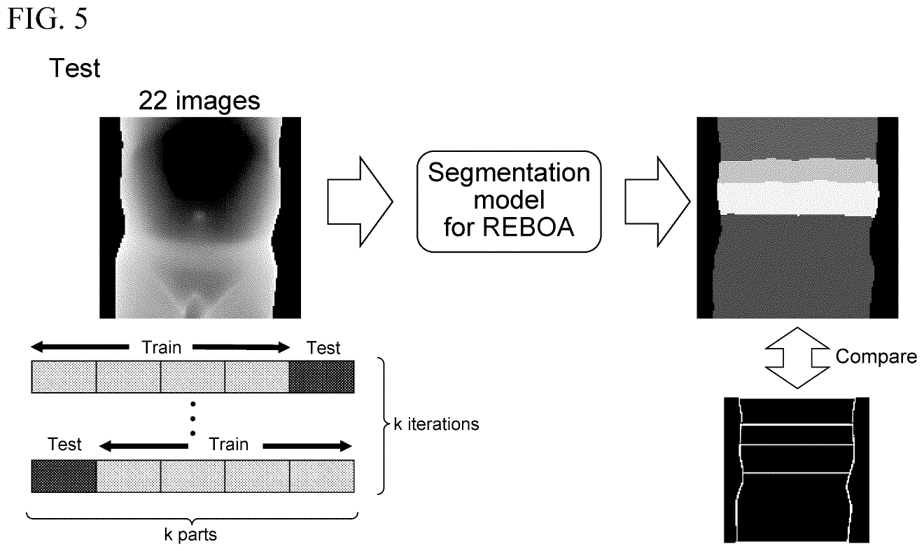

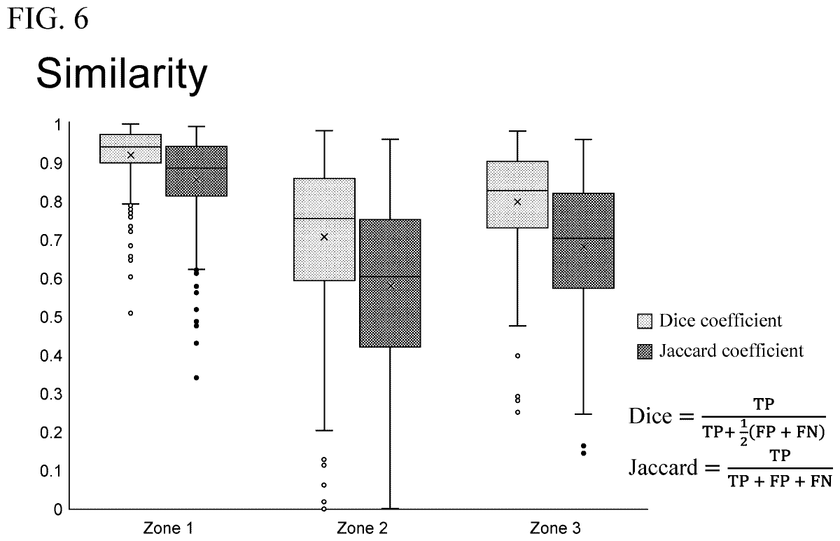

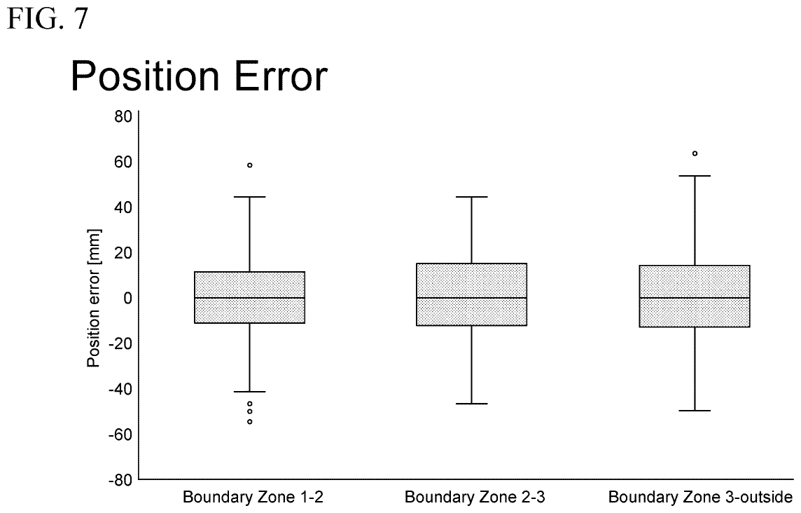

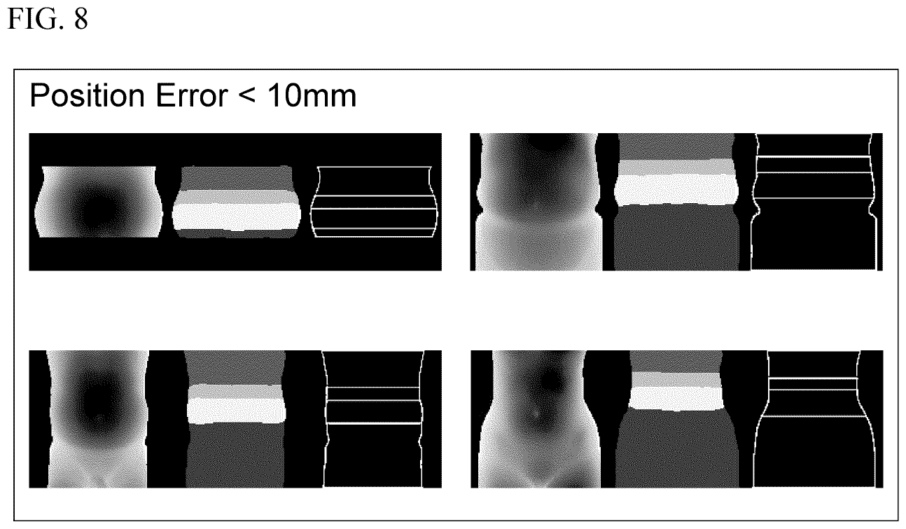

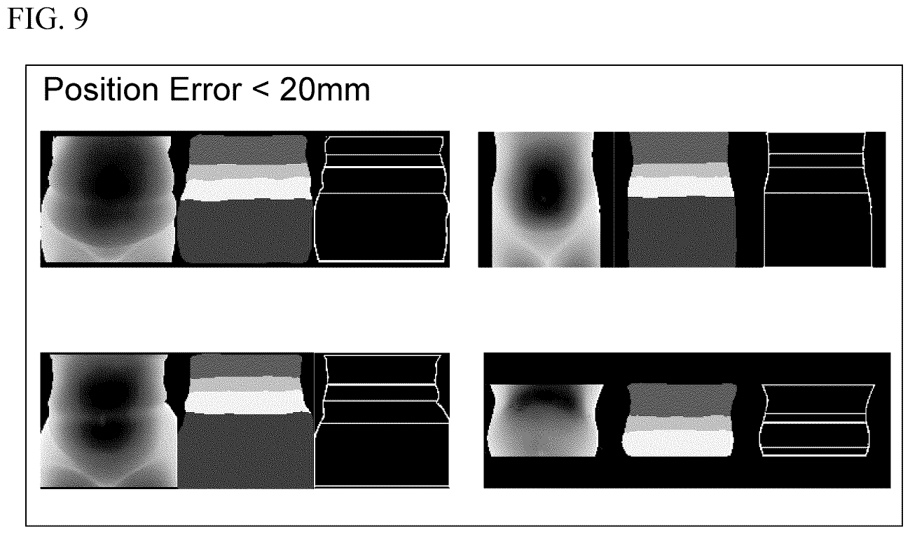

FIG. 4 is a diagram conceptually showing training of a semantic segmentation model using the training data set. FIG. 5 is a diagram conceptually showing a test of the trained semantic segmentation model. FIG. 6 is a diagram showing a result of evaluating, using a Dice coefficient and a Jaccard coefficient, a similarity between Zone 1, Zone 2, and Zone 3 estimated using the trained semantic segmentation model. FIG. 7 is a diagram showing a position error of a boundary line between Zone 1 and Zone 2, a position error of a boundary line between Zone 2 and Zone 3, and the like, which are estimated using the trained semantic segmentation model. FIG. 8 is a diagram showing four examples in which a position error of the aortic segment estimated using the trained semantic segmentation model is less than 10 mm. FIG. 9 is a diagram showing four examples in which a position error of the aortic segment estimated using the trained semantic segmentation model is larger than 20 mm.

Further, the present inventor performed the following in order to develop an application that can intuitively visualize a Zone estimated using a trained model, using mixed reality that seamlessly combines reality and virtuality.

In the research, the present inventor developed an application that operates on an iPad Pro (“iPad” is a registered trademark) (manufactured by Apple Inc.), which is a tablet-type terminal, and estimated and visualized a Zone from a trained model. The iPad Pro includes an infrared camera and can perform three-dimensional measurement of real space. Three-dimensional information of a patient body surface imaged by the infrared camera is input to the trained model, and the estimation result of the Zone is projected in real time onto the body surface imaged by a normal camera. As a result, the user of the blood vessel segment discrimination system 1 visualizes the Zone segment only by holding the iPad Pro over the human body. In this research, the iPad Pro functions as the three-dimensional structure recognition device 11 and as the visualization device 15.

In addition, in the research, the present inventor developed an application that operates on HoloLens2 (“HOLOLENS” is an international registered trademark) (manufactured by Microsoft Corporation) which is a headset-type terminal, and estimates and visualizes a Zone from a trained model. The HoloLens 2 also includes an infrared camera and can perform three-dimensional measurement of real space, as in the iPad Pro. Three-dimensional information of the body surface is used as an input to the trained model, and the estimation result of the Zone is projected in real time onto the body surface visible through the see-through HoloLens 2. As a result, the user of the blood vessel segment discrimination system 1 visualizes the Zone segment only by wearing the HoloLens 2 and looking at the patient. In this research, the HoloLens 2 functions as the three-dimensional structure recognition device 11 and as the visualization device 15.

Since an operation system and a hardware configuration of the HoloLens2 are different from those of the iPad Pro, it is necessary to change a surface layer portion of the application, but a portion related to the Zone segment estimation, which is a core of the system, can be shared (that is, either the iPad Pro or the HoloLens2 can be used as the visualization device 15).

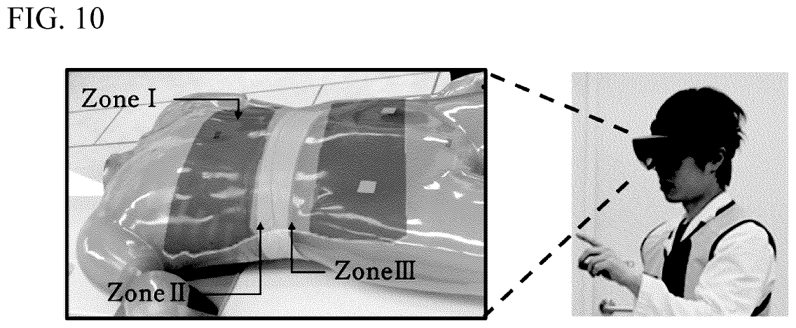

FIG. 10 is a diagram conceptually describing a technique of projecting the estimation result of the Zone onto the body surface of a patient in real time using the HoloLens 2.

As a result of the research described above, the present inventor found that the Zone segment can be estimated with sufficiently high accuracy from a three-dimensional image of the body surface of a patient. Specifically, the present inventor constructed a data set from an extremely small part of the CT image and performed a test of segmentation by deep learning. As a result, the reproducibility of Zone 1 was maximized to 0.99, the reproducibility of Zone 2 was maximized to 0.95, and the reproducibility of Zone 3 was maximized to 0.98.

The present inventor studied an implementation method of visualization using mixed reality based on the knowledge acquired so far, and realized a technique of projecting the Zone onto the body surface of a patient (that is, operating an actual device as the blood vessel segment discrimination system 1).

### Second Embodiment

Hereinafter, a second embodiment of the blood vessel segment discrimination system, the blood vessel segment discrimination method, and the program according to the present invention will be described.

The blood vessel segment discrimination system 1 according to the second embodiment is configured in the same manner as the blood vessel segment discrimination system 1 according to the first embodiment described above, except for the following points. Therefore, with the blood vessel segment discrimination system 1 of the second embodiment, the same effects as those of the blood vessel segment discrimination system 1 of the first embodiment described above can be obtained, except for the following points.

As described above, in the blood vessel segment discrimination system 1 according to the first embodiment, the segmentation model is used as the deep learning model 14A.

On the other hand, in the blood vessel segment discrimination system 1 of the second embodiment, as the deep learning model 14A, a pseudo image generation technique such as a generative adversarial network (GAN) described on, for example, the following websites is used.
https://ledge.ai/gan/
https://www.nedo.go.jp/news/press/AA5_101472.html

### Third Embodiment

Hereinafter, a third embodiment of the blood vessel segment discrimination system, the blood vessel segment discrimination method, and the program according to the present invention will be described.

A blood vessel segment discrimination system 2 according to the third embodiment is configured in the same manner as the blood vessel segment discrimination system 1 according to the first embodiment described above, except for the following points. Therefore, with the blood vessel segment discrimination system 2 according to the third embodiment, the same effects as those of the blood vessel segment discrimination system 1 according to the first embodiment described above can be obtained, except for the following points.

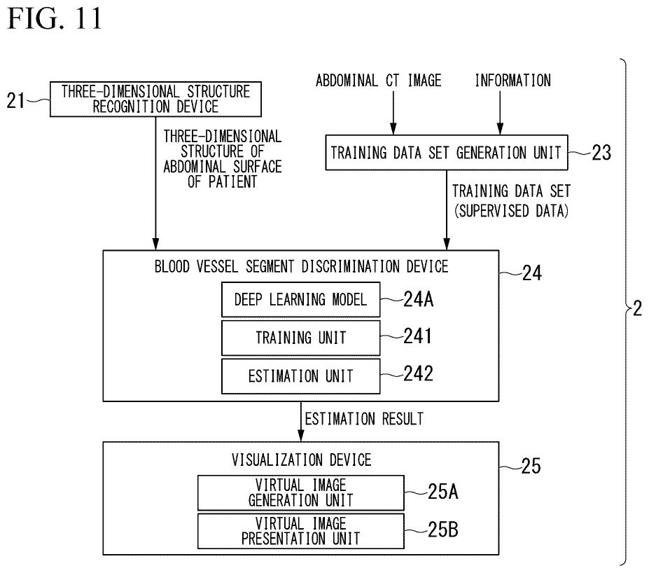

FIG. 11 is a diagram showing an example of the blood vessel segment discrimination system 2 according to the third embodiment.

In the example shown in FIG. 11, the blood vessel segment discrimination system 2 of the third embodiment supports the implementation of REBOA on the patient by the operator in a situation in which the X-ray fluoroscopy or ultrasound device cannot be used, for example, in an emergency, in the same manner as the blood vessel segment discrimination system 1 of the first embodiment. The blood vessel segment discrimination system 2 includes a three-dimensional structure recognition device 21, a training data set generation unit 23, a blood vessel segment discrimination device 24, and a visualization device 25.

The three-dimensional structure recognition device 21 recognizes a three-dimensional structure of the abdomen of the patient in the same manner as the three-dimensional structure recognition device 11 shown in FIG. 1.

The training data set generation unit 23 generates a training data set.

The blood vessel segment discrimination device 24 discriminates the aortic segment of the patient whose three-dimensional structure of the abdominal surface is recognized by the three-dimensional structure recognition device 21 using a deep learning model 24A.

In the example of the blood vessel segment discrimination system 2 according to the third embodiment, the training data set generation unit 23 generates the three-dimensional structure of the abdominal surface for training from any of the abdominal CT image, the abdominal MRI image, and the abdominal MRA image of a person different from a patient who is a discrimination target of the aortic segment by the blood vessel segment discrimination device 24.

Further, the training data set generation unit 23 generates information showing a correspondence relationship between each point on the three-dimensional structure of the abdominal surface for training and any of the first blood vessel segment corresponding to Zone 1 of the aorta, the second blood vessel segment corresponding to Zone 2 of the aorta, the third blood vessel segment corresponding to Zone 3 of the aorta, and another segment, based on any of the abdominal CT image, the abdominal MRI image, and the abdominal MRA image.

In addition, the training data set generation unit 23 generates a training data set, which is a set of the three-dimensional structure of the abdominal surface for training and the information described above, as supervised data used for training the deep learning model 24A.

In the example shown in FIG. 11, the blood vessel segment discrimination device 24 includes a training unit 241 and an estimation unit 242. The training unit 241 performs training of the deep learning model 24A using the training data set generated by the training data set generation unit 23.

Specifically, since the risk of organ ischemia increases if an estimation accuracy of the second blood vessel segment corresponding to Zone 2 of the aorta is low in a case where REBOA is performed, the training unit 241 performs training of the deep learning model 24A such that the estimation accuracy of the second blood vessel segment corresponding to Zone 2 of the aorta using the trained deep learning model 24A is equal to or higher than a predetermined threshold value.

In the blood vessel segment discrimination system 2 according to the third embodiment, the segmentation model is used as the deep learning model 24A, in the same manner as in the blood vessel segment discrimination system 1 according to the first embodiment.

Specifically, in a first example of the blood vessel segment discrimination system 1 according to the third embodiment, the semantic segmentation model described above is used as the deep learning model 24A.

In a second example of the blood vessel segment discrimination system 2 according to the third embodiment, as the deep learning model 24A, a segmentation model other than semantic segmentation, such as instance segmentation and panoptic segmentation described above, may be used.

In the example shown in FIG. 11, the estimation unit 242 uses the trained deep learning model 24A to estimate whether each point on the three-dimensional structure of the patient recognized by the three-dimensional structure recognition device 21 corresponds to any of the first blood vessel segment corresponding to Zone 1 of the aorta, the second blood vessel segment corresponding to Zone 2 of the aorta, the third blood vessel segment corresponding to Zone 3 of the aorta, and another segment (that is, a segment that does not correspond to any of the first blood vessel segment, the second blood vessel segment, and the third blood vessel segment).

That is, in the example shown in FIG. 11, the three-dimensional structure recognition device 21 recognizes the three-dimensional structure of the abdomen of the patient after the deep learning model 24A is trained using the training data set generated by the training data set generation unit 23. In addition, the estimation unit 242 of the blood vessel segment discrimination device 24 uses the trained deep learning model 24A to estimate whether each point on the three-dimensional structure of the patient recognized by the three-dimensional structure recognition device 21 corresponds to any of the first blood vessel segment corresponding to Zone 1 of the aorta, the second blood vessel segment corresponding to Zone 2 of the aorta, the third blood vessel segment corresponding to Zone 3 of the aorta, and another segment.

The visualization device 25 generates a virtual image in which the estimation result of the aortic segment of the patient by the blood vessel segment discrimination device 24 is projected onto the abdomen of the patient. The visualization device 25 includes a virtual image generation unit 25A and a virtual image presentation unit 25B.

The virtual image generation unit 25A generates a virtual image in which the first blood vessel segment (Zone 1), the second blood vessel segment (Zone 2), and the third blood vessel segment (Zone 3) of the patient are projected onto the abdominal surface of the patient. The virtual image presentation unit 25B presents the virtual image generated by the virtual image generation unit 25A to the user of the blood vessel segment discrimination system 2.

In the first example of the blood vessel segment discrimination system 2 according to the third embodiment, the visualization device 25 projects the first blood vessel segment (Zone 1), the second blood vessel segment (Zone 2), and the third blood vessel segment (Zone 3) of the patient onto the abdomen of the patient, in the same manner as in the first example of the blood vessel segment discrimination system 1 according to the first embodiment.

In the second example of the blood vessel segment discrimination system 2 according to the third embodiment, the visualization device 25 directly uses a real three-dimensional object (that is, the abdomen of the patient) as a marker, that is, directly projects the first blood vessel segment (Zone 1), the second blood vessel segment (Zone 2), and the third blood vessel segment (Zone 3) of the patient onto the abdomen of the patient without any external intervention, in the same manner as in the second example of the blood vessel segment discrimination system 1 according to the first embodiment.

In a third example of the blood vessel segment discrimination system 2 according to the third embodiment, the visualization device 25 directly uses the real three-dimensional object (that is, the abdomen of the patient) as a marker, that is, directly projects the first blood vessel segment (Zone 1), the second blood vessel segment (Zone 2), and the third blood vessel segment (Zone 3) of the patient onto the abdomen of the patient without any external intervention, in the same manner as in the third example of the blood vessel segment discrimination system 1 according to the first embodiment.

In the third example of the blood vessel segment discrimination system 2 according to the third embodiment, the visualization device 25 generates a virtual image in which the first blood vessel segment (Zone 1), the second blood vessel segment (Zone 2), and the third blood vessel segment (Zone 3) of the patient are projected onto the abdominal surface of the patient, and presents the virtual image to the user of the blood vessel segment discrimination system 2, in the same manner as in the third example of the blood vessel segment discrimination system 1 according to the first embodiment.

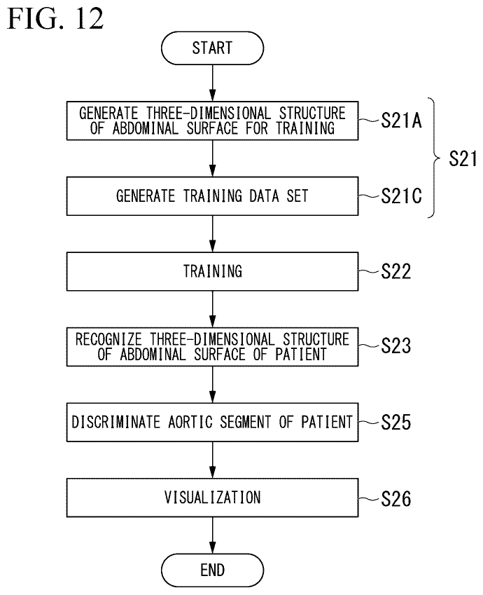

FIG. 12 is a flowchart describing an example of processing executed in the blood vessel segment discrimination system 2 according to the third embodiment.

In the example shown in FIG. 12, in step S21, the training data set generation unit 23 generates the training data set used for the training of the deep learning model 24A.

Specifically, in step S21A, the training data set generation unit 23 generates the three-dimensional structure of the abdominal surface for training from any of the abdominal CT image, the abdominal MRI image, and the abdominal MRA image of a person different from a patient who is a discrimination target of the aortic segment by the blood vessel segment discrimination device 14. Next, in step S21C, the training data set generation unit 13 generates the training data set showing a correspondence relationship between each point on the three-dimensional structure of the abdominal surface for training and any of the first blood vessel segment corresponding to Zone 1 of the aorta, the second blood vessel segment corresponding to Zone 2 of the aorta, the third blood vessel segment corresponding to Zone 3 of the aorta, and another segment (that is, a segment that does not correspond to any of the first blood vessel segment, the second blood vessel segment, and the third blood vessel segment) based on any of the abdominal CT image, the abdominal MRI image, and the abdominal MRA image.

Next, in step S22, the training unit 241 of the blood vessel segment discrimination device 24 performs training of the deep learning model 24A using the training data set generated in step S21.

Next, in step S23, the three-dimensional structure recognition device 21 recognizes the three-dimensional structure of the abdomen of the patient who is the discrimination target of the aortic segment by the blood vessel segment discrimination device 24.

Next, in step S25, the blood vessel segment discrimination device 14 discriminates the aortic segment of the patient whose three-dimensional structure of the abdominal surface is recognized in step S23 using the trained deep learning model 24A that has been trained in step S22.

Specifically, in step S25, the estimation unit 242 of the blood vessel segment discrimination device 24 uses the trained deep learning model 24A to estimate whether each point on the three-dimensional structure of the abdomen of the patient recognized in step S23 corresponds to any of the first blood vessel segment corresponding to Zone 1of the aorta, the second blood vessel segment corresponding to Zone 2 of the aorta, the third blood vessel segment corresponding to Zone 3 of the aorta, and another segment (that is, a segment that does not correspond to any of the first blood vessel segment, the second blood vessel segment, and the third blood vessel segment).

Next, in step S26, the virtual image generation unit 25A of the visualization device 25 generates a virtual image in which the first blood vessel segment (Zone 1), the second blood vessel segment (Zone 2), and the third blood vessel segment (Zone 3) of the patient are projected onto the abdominal surface of the patient. In addition, the virtual image presentation unit 25B of the visualization device 25 presents the virtual image generated by the virtual image generation unit 25A to the user of the blood vessel segment discrimination system 2.

Therefore, with the blood vessel segment discrimination system 2 according to the third embodiment, the user of the blood vessel segment discrimination system 2 can easily identify Zone 1, Zone 2, and Zone 3 of the aorta of the patient even in a situation in which the X-ray fluoroscopy or ultrasound device cannot be used. As a result, it is possible to improve the availability of REBOA in a situation in which the X-ray fluoroscopy or ultrasound device cannot be used.

As described above, in the example shown in FIG. 11, the three-dimensional structure recognition device 21 recognizes the three-dimensional structure of the abdomen of the patient who is the discrimination target of the aortic segment by the blood vessel segment discrimination device 24 and generates the three-dimensional data of the abdomen of the patient using a technique similar to the LiDAR scanner. In another example, as in the example shown in FIG. 2, the three-dimensional structure recognition device 21 may have a function of generating the three-dimensional structure of the abdomen of the patient from any of the abdominal CT image, the abdominal MRI image, and the abdominal MRA image of the patient who is the discrimination target of the aortic segment by the blood vessel segment discrimination device 24.

As described above, in the example shown in FIG. 11, the blood vessel segment discrimination device 24 outputs, as the estimation result of the aortic segment of the patient, information used by the visualization device 25 to generate the virtual image in which the first blood vessel segment (Zone 1), the second blood vessel segment (Zone 2), and the third blood vessel segment (Zone 3) are projected onto the abdomen of the patient.

In another example, the blood vessel segment discrimination device 24 may output, as the estimation result of the aortic segment of the patient, a length from a landmark part (that is, a site suitable for puncture (that is, a catheter insertion site)) of the patient to the first blood vessel segment (Zone 1) of the patient, a length from the landmark part of the patient to the second blood vessel segment (Zone 2) of the patient, and a length from the landmark part of the patient to the third blood vessel segment (Zone 3) of the patient, for example, in numerical values or like.

### Fourth Embodiment

Hereinafter, a fourth embodiment of the blood vessel segment discrimination system, the blood vessel segment discrimination method, and the program according to the present invention will be described.

A blood vessel segment discrimination system 2 according to the fourth embodiment is configured in the same manner as the blood vessel segment discrimination system 2 according to the third embodiment described above, except for the following points. Therefore, with the blood vessel segment discrimination system 2 according to the fourth embodiment, the same effects as those of the blood vessel segment discrimination system 2 according to the third embodiment described above can be obtained, except for the following points.

As described above, in the blood vessel segment discrimination system 2 according to the third embodiment, the segmentation model is used as the deep learning model 24A.

On the other hand, in the blood vessel segment discrimination system 2 according to the fourth embodiment, as the deep learning model 24A, a pseudo image generation technique such as the GAN described above is used.

Hitherto, the embodiments of the present invention have been described in detail with reference to the drawings. A specific configuration is not limited to the embodiments, and can be appropriately modified within the scope not departing from the concept of the present invention. The configurations described above in each of the embodiments and each of the examples may be combined.

An entirety or a part of the blood vessel segment discrimination system 1 or 2 in the embodiments described above may be realized by dedicated hardware, or may be realized by a memory and a microprocessor.

An entirety or a part of the blood vessel segment discrimination system 1 may include a memory and a central processing unit (CPU), and may realize a function thereof in such a manner that a program for realizing a function of each unit included in each system is loaded to and executed by the memory.

A program for realizing all or a part of the functions of the blood vessel segment discrimination system 1 may be recorded on a computer-readable recording medium, and the program recorded on the recording medium may be read and executed by a computer system to perform processing of each unit. The “computer system” referred to herein includes an OS and hardware such as a peripheral device. In addition, the “computer system” also includes a homepage providing environment (or a display environment) in a case where a WWW system is used.

In addition, the “computer-readable recording medium” refers to a portable medium such as a flexible disk, a magneto-optical disk, a ROM, and a CD-ROM, and a storage device such as a hard disk built in the computer system. Furthermore, the “computer-readable recording medium” includes a medium which dynamically holds the program for a short period of time as in a communication line in a case where the program is transmitted through a network such as the Internet or a communication line such as a telephone line, and a medium which holds the program for a certain period of time as in a volatile memory inside the computer system serving as a server or a client in that case. In addition, the program may be provided to realize a part of the above-described functions, or may be provided to be capable of realizing the above-described functions in combination with a program already recorded on the computer system.

### REFERENCE SIGNS LIST

1: Blood vessel segment discrimination system
11: Three-dimensional structure recognition device
12: Depth image generation unit
13: Training data set generation unit
14: Blood vessel segment discrimination device
14A: Deep learning model
141: Training unit
142: Estimation unit
15: Visualization device
15A: Virtual image generation unit
15B: Virtual image presentation unit
2: Blood vessel segment discrimination system
21: Three-dimensional structure recognition device
23: Training data set generation unit
24: Blood vessel segment discrimination device
24A: Deep learning model
241: Training unit
242: Estimation unit
25: Visualization device
25A: Virtual image generation unit
25B: Virtual image presentation unit

1. A blood vessel segment discrimination system, comprising:
a three-dimensional structure recognition device configured to recognize a three-dimensional structure of an abdomen of the patient;
a depth image generation unit configured to generate a depth image of the abdomen of the patient from the three-dimensional structure of the abdominal surface of the patient recognized by the three-dimensional structure recognition device;
a blood vessel segment discrimination device configured to discriminate an aortic segment of the patient whose three-dimensional structure of the abdominal surface is recognized by the three-dimensional structure recognition device, using a deep learning model; and
a training data set generation unit configured to generate a training data set used for training of the deep learning model,
wherein, before the training of the deep learning model is performed, the training data set
generation unit
generates the three-dimensional structure of the abdominal surface for training from any of an abdominal computed tomography (CT) image, an abdominal magnetic resonance imaging (MRI) image, and an abdominal magnetic resonance angiography (MRA) image of a person different from a patient who is a discrimination target of the aortic segment by the blood vessel segment discrimination device,
generates a depth image for training from the three-dimensional structure of the abdominal surface for training, and
generates the training data set showing a correspondence relationship between each pixel in the depth image for training and any of a first blood vessel segment corresponding to a Zone 1 of an aorta, a second blood vessel segment corresponding to a Zone 2 of the aorta, a third blood vessel segment corresponding to a Zone 3 of the aorta, and another segment, based on any of the abdominal CT image, the abdominal MRI image, and the abdominal MRA image, and
after the training of the deep learning model is performed using the training data set generated by the training data set generation unit,
the three-dimensional structure recognition device recognizes the three-dimensional structure of the abdomen of the patient,
the depth image generation unit generates the depth image of the abdomen of the patient from the three-dimensional structure of the abdominal surface of the patient recognized by the three-dimensional structure recognition device, and
the blood vessel segment discrimination device estimates whether each pixel in the depth image of the abdomen of the patient generated by the depth image generation unit corresponds to any of the first blood vessel segment, the second blood vessel segment, the third blood vessel segment, and another segment using the trained deep learning model.

2. The blood vessel segment discrimination system according to claim 1, further comprising:
a visualization device configured to generate a virtual image in which an estimation result of the aortic segment of the patient by the blood vessel segment discrimination device is projected onto the abdomen of the patient.

3. The blood vessel segment discrimination system according to claim 1,
wherein the blood vessel segment discrimination device includes
a training unit configured to perform the training of the deep learning model using the training data set generated by the training data set generation unit, and
an estimation unit configured to estimate whether each pixel in the depth image of the abdomen of the patient generated by the depth image generation unit corresponds to any of the first blood vessel segment, the second blood vessel segment, the third blood vessel segment, and another segment, using the trained deep learning model, and
the training unit performs the training of the deep learning model such that an estimation accuracy of the second blood vessel segment using the trained deep learning model is equal to or higher than a predetermined threshold value.

4. The blood vessel segment discrimination system according to claim 1,
wherein the three-dimensional structure recognition device has a function of generating the three-dimensional structure of the abdomen of the patient from any of the abdominal CT image, the abdominal MRI image, and the abdominal MRA image of the patient.

5. The blood vessel segment discrimination system according to claim 1,
wherein the blood vessel segment discrimination device outputs, as an estimation result of the aortic segment of the patient, a length from a landmark part of the patient to the first blood vessel segment of the patient, a length from the landmark part of the patient to the second blood vessel segment of the patient, and a length from the landmark part of the patient to the third blood vessel segment of the patient.

6. A blood vessel segment discrimination method for a blood vessel segment discrimination system including:
a three-dimensional structure recognition device configured to recognize a three-dimensional structure of an abdomen of the patient;
a depth image generation unit configured to generate a depth image of the abdomen of the patient from the three-dimensional structure of the abdominal surface of the patient recognized by the three-dimensional structure recognition device;
a blood vessel segment discrimination device configured to discriminate an aortic segment of the patient whose three-dimensional structure of the abdominal surface is recognized by the three-dimensional structure recognition device, using a deep learning model; and
a training data set generation unit configured to generate a training data set used for training of the deep learning model, the blood vessel segment discrimination method comprising:
a training data set generation step of, before the training of the deep learning model is performed, via the training data set generation unit,
generating the three-dimensional structure of the abdominal surface for training from any of an abdominal CT image, an abdominal MRI image, and an abdominal MRA image of a person different from a patient who is a discrimination target of the aortic segment by the blood vessel segment discrimination device,
generating a depth image for training from the three-dimensional structure of the abdominal surface for training, and
generating the training data set showing a correspondence relationship between each pixel in the depth image for training and any of a first blood vessel segment corresponding to a Zone 1 of an aorta, a second blood vessel segment corresponding to a Zone 2 of the aorta, a third blood vessel segment corresponding to a Zone 3 of the aorta, and another segment, based on any of the abdominal CT image, the abdominal MRI image, and the abdominal MRA image;
a three-dimensional structure recognition step of, after the training of the deep learning model is performed using the training data set generated in the training data set generation step, via the three-dimensional structure recognition device, recognizing the three-dimensional structure of the abdomen of the patient;
a depth image generation step of, via the depth image generation unit, generating the depth image of the abdomen of the patient from the three-dimensional structure of the abdominal surface of the patient recognized in the three-dimensional structure recognition step; and
a blood vessel segment discrimination step of, via the blood vessel segment discrimination device, estimating whether each pixel in the depth image of the abdomen of the patient generated in the depth image generation step corresponds to any of the first blood vessel segment, the second blood vessel segment, the third blood vessel segment, and another segment using the trained deep learning model.

7. A program for causing a computer constituting a blood vessel segment discrimination device provided in a blood vessel segment discrimination system including:
a three-dimensional structure recognition device configured to recognize a three-dimensional structure of an abdomen of the patient;
a depth image generation unit configured to generate a depth image of the abdomen of the patient from the three-dimensional structure of the abdominal surface of the patient recognized by the three-dimensional structure recognition device; and
a training data set generation unit configured to generate a training data set used for training of a deep learning model, to execute:
a training step of performing the training of the deep learning model using the training data set generated by the training data set generation unit; and
a blood vessel segment discrimination step,
wherein the training data set generation unit
generates the three-dimensional structure of the abdominal surface for training from any of an abdominal CT image, an abdominal MRI image, and an abdominal MRA image of a person different from a patient who is a discrimination target of an aortic segment by the blood vessel segment discrimination device,
generates a depth image for training from the three-dimensional structure of the abdominal surface for training, and generates the training data set showing a correspondence relationship between each pixel in the depth image for training and any of a first blood vessel segment corresponding to a Zone 1of an aorta, a second blood vessel segment corresponding to a Zone 2 of the aorta, a third blood vessel segment corresponding to a Zone 3 of the aorta, and another segment, based on any of the abdominal CT image, the abdominal MRI image, and the abdominal MRA image,
after the training step is executed, the three-dimensional structure recognition device recognizes the three-dimensional structure of the abdomen of the patient, and the depth image generation unit generates the depth image of the abdomen of the patient from the three-dimensional structure of the abdominal surface of the patient recognized by the three-dimensional structure recognition device, and
in the blood vessel segment discrimination step, estimation is made as to whether each pixel in the depth image of the abdomen of the patient generated by the depth image generation unit corresponds to any of the first blood vessel segment, the second blood vessel segment, the third blood vessel segment, and another segment using the trained deep learning model.

8. A blood vessel segment discrimination system, comprising:
a three-dimensional structure recognition device configured to recognize a three-dimensional structure of an abdomen of the patient;
a blood vessel segment discrimination device configured to discriminate an aortic segment of the patient whose three-dimensional structure of the abdominal surface is recognized by the three-dimensional structure recognition device, using a deep learning model; and
a training data set generation unit configured to generate a training data set used for training of the deep learning model,
wherein, before the training of the deep learning model is performed, the training data set generation unit
generates the three-dimensional structure of the abdominal surface for training from any of an abdominal CT image, an abdominal MRI image, and an abdominal MRA image of a person different from a patient who is a discrimination target of the aortic segment by the blood vessel segment discrimination device, and
generates the training data set showing a correspondence relationship between each point on the three-dimensional structure of the abdominal surface for training and any of a first blood vessel segment corresponding to a Zone 1 of an aorta, a second blood vessel segment corresponding to a Zone 2 of the aorta, a third blood vessel segment corresponding to a Zone 3 of the aorta, and another segment, based on any of the abdominal CT image, the abdominal MRI image, and the abdominal MRA image, and
after the training of the deep learning model is performed using the training data set generated by the training data set generation unit,
the three-dimensional structure recognition device recognizes the three-dimensional structure of the abdomen of the patient, and
the blood vessel segment discrimination device estimates whether each point on the three-dimensional structure of the abdomen of the patient recognized by the three-dimensional structure recognition device corresponds to any of the first blood vessel segment, the second blood vessel segment, the third blood vessel segment, and another segment using the trained deep learning model.

9. The blood vessel segment discrimination system according to claim 8, further comprising:
a visualization device configured to generate a virtual image in which an estimation result of the aortic segment of the patient by the blood vessel segment discrimination device is projected onto the abdomen of the patient.

10. The blood vessel segment discrimination system according to claim 8,
wherein the blood vessel segment discrimination device includes
a training unit configured to perform the training of the deep learning model using the training data set generated by the training data set generation unit, and
an estimation unit configured to estimate whether each point on the three-dimensional structure of the abdomen of the patient recognized by the three-dimensional structure recognition device corresponds to any of the first blood vessel segment, the second blood vessel segment, the third blood vessel segment, and another segment, using the trained deep learning model, and
the training unit performs the training of the deep learning model such that an estimation accuracy of the second blood vessel segment using the trained deep learning model is equal to or higher than a predetermined threshold value.

11. The blood vessel segment discrimination system according to claim 8,
wherein the three-dimensional structure recognition device has a function of generating the three-dimensional structure of the abdomen of the patient from any of the abdominal CT image, the abdominal MRI image, and the abdominal MRA image of the patient.

12. The blood vessel segment discrimination system according to claim 8,
wherein the blood vessel segment discrimination device outputs, as an estimation result of the aortic segment of the patient, a length from a landmark part of the patient to the first blood vessel segment of the patient, a length from the landmark part of the patient to the second blood vessel segment of the patient, and a length from the landmark part of the patient to the third blood vessel segment of the patient.

13. A blood vessel segment discrimination method for a blood vessel segment discrimination system including:
a three-dimensional structure recognition device configured to recognize a three-dimensional structure of an abdomen of the patient;
a blood vessel segment discrimination device configured to discriminate an aortic segment of the patient whose three-dimensional structure of the abdominal surface is recognized by the three-dimensional structure recognition device, using a deep learning model; and
a training data set generation unit configured to generate a training data set used for training of the deep learning model, the blood vessel segment discrimination method comprising:
a training data set generation step of, before the training of the deep learning model is performed, via the training data set generation unit,
generating the three-dimensional structure of the abdominal surface for training from any of an abdominal CT image, an abdominal MRI image, and an abdominal MRA image of a person different from a patient who is a discrimination target of the aortic segment by the blood vessel segment discrimination device, and
generating the training data set showing a correspondence relationship between each point on the three-dimensional structure of the abdominal surface for training and any of a first blood vessel segment corresponding to a Zone 1 of an aorta, a second blood vessel segment corresponding to a Zone 2 of the aorta, a third blood vessel segment corresponding to a Zone 3 of the aorta, and another segment, based on any of the abdominal CT image, the abdominal MRI image, and the abdominal MRA image;
a three-dimensional structure recognition step of, after the training of the deep learning model is performed using the training data set generated in the training data set generation step, via the three-dimensional structure recognition device, recognizing the three-dimensional structure of the abdomen of the patient; and
a blood vessel segment discrimination step of, via the blood vessel segment discrimination device, estimating whether each point on the three-dimensional structure of the abdomen of the patient recognized in the three-dimensional structure recognition step corresponds to any of the first blood vessel segment, the second blood vessel segment, the third blood vessel segment, and another segment using the trained deep learning model.

14. A program for causing a computer constituting a blood vessel segment discrimination device provided in a blood vessel segment discrimination system including:
a three-dimensional structure recognition device configured to recognize a three-dimensional structure of an abdomen of the patient; and
a training data set generation unit configured to generate a training data set used for training of a deep learning model, to execute:
a training step of performing the training of the deep learning model using the training data set generated by the training data set generation unit; and
a blood vessel segment discrimination step,
wherein the training data set generation unit
generates the three-dimensional structure of the abdominal surface for training from any of an abdominal CT image, an abdominal MRI image, and an abdominal MRA image of a person different from a patient who is a discrimination target of an aortic segment by the blood vessel segment discrimination device, and
generates the training data set showing a correspondence relationship between each point on the three-dimensional structure of the abdominal surface for training and any of a first blood vessel segment corresponding to a Zone 1 of an aorta, a second blood vessel segment corresponding to a Zone 2 of the aorta, a third blood vessel segment corresponding to a Zone 3 of the aorta, and another segment, based on any of the abdominal CT image, the abdominal MRI image, and the abdominal MRA image,
after the training step is executed, the three-dimensional structure recognition device recognizes the three-dimensional structure of the abdomen of the patient, and
in the blood vessel segment discrimination step, estimation is made as to whether each point on the three-dimensional structure of the abdomen of the patient recognized by the three-dimensional structure recognition device corresponds to any of the first blood vessel segment, the second blood vessel segment, the third blood vessel segment, and another segment using the trained deep learning model.
